(19) United States
(12) Patent Application Publication
(10) Pub. No.: US 2009/0036413 A1
(43) Pub. Date: Feb. 5, 2009

(54) Compositions, uses, and method of making wound care products from naturally occurring food ingredients

(75) Inventors: Shayne McAnalley, Cedar Hill, TX (US); Bill McAnalley, Grand Prairie, TX (US); Erik Aguayo, Everman, TX (US)

Correspondence Address: JACKSON WALKER LLP, 901 MAIN STREET, SUITE 6000, DALLAS, TX, 75202-3797, US

(73) Assignee: Bill McAnalley, Grand Prairie, TX (US)

(21) Appl. No.: 12/220,854

(22) Filed: Jul. 29, 2008

Related U.S. Application Data
(60) Provisional application No. 60/962,676, filed on Jul. 31, 2007.

(51) Int. Cl. A61K 31/60; A61K 31/11; A61P 17/02

(52) U.S. Cl. 514/163; 514/701

(57) ABSTRACT

Rationally designed wound care products made entirely of naturally occurring food ingredients that can be standardized and made available for the mass market using good manufacturing practice (GMP) guidelines, optionally, a safe food additive can be added. These products: are safe and effective; have an osmotic pressure compatible with optimal healing; are buffered to maintain optimal pH throughout the healing process; provide a protective barrier from further irritation and insult; control bacteria, viruses and fungi found in the skin and mucosa; nourish wounds; control excessive prolonged inflammation and thereby minimize scarring; minimize allergenic and irritation potential; are easy to use or apply; pass the preservative challenge test required for products intended for multiple use; contain fragrant essential oils to take advantage of the benefits provided by aromatherapy; can be individually optimized based on the diet of an individual or group of people.

This application claims priority to U.S. Provisional Patent Application No. 60/962,676, filed Jul. 31, 2007, entitled “COMPOSITIONS, USES, AND METHOD OF MAKING WOUND CARE PRODUCTS FROM NATURALLY OCCURRING FOOD INGREDIENTS”, the entire content of which is hereby incorporated by reference.

### BACKGROUND

This invention pertains to wound care products made principally from food ingredients and, optionally, with the addition of safe food additives, their preparation and uses.

### I The State of the Art of Wound Care Products

The state of the art is well described by Liza G. Ovington, PhD, in an article titled “Advances in Wound Dressings.”¹ Moist wound healing is the most significant advance in the concept of wound healing since sterile technique and materials were championed by Semmelweiss and Pasteur in the 19^{th} century.¹ Currently, dressings providing optimal hydration fall into three broad categories described by Ovington: some absorb excessive exudate from the wound, some maintain the level of moisture, and some add moisture. “Moist wound dressings are also referred to as occlusive or semiocclusive dressings, advanced dressings, or modern dressings and are quite heterogeneous.” Currently there are more than 400 wound dressings that fall into these categories.²

New products are being developed for chronic, non-healing wounds. One target of these new wound healing products is matrix metalloproteases (MMPs). MMPs act in cellular migration. In wounds there is a natural increase in the level of MMPs during the inflammatory phase of wound healing. In normal wound healing, this is a self-limited step. It has been shown, however, that in non-healing wounds, particularly bacterially infected wounds, MMPs are at elevated levels. A new wound dressing containing 55% bovine collagen and 45% oxidized regenerated cellulose is being used to counter this effect by chemically binding the MMP family of enzymes rendering them inactive. The oxidized regenerated cellulose moiety has multiple negative charges. These negative charges dislodge the positively charged metal ions common to the oxidized regenerated cellulose moiety, thus inactivating these enzymes. A new sulfonated polymer wound dressing has been developed that binds and inactivates proteases as well.¹

“Recently, dressings that contain and release antimicrobial agents at the wound surface have entered the market. These dressings usually provide a continuous or sustained release of the antiseptic agent.” The current antiseptic products on the market include ionic silver, molecular iodine, and polyhexamethyl biguanide (PHMB).¹ Future advances in wound dressings are the combination of current wound dressings with the addition of drugs. However, a result of the recent, surprising finding that many people have developed allergies to bacitracin, a drug ingredient commonly used in wound dressings³, this could move this type of wound dressing from being regulated as a device into being regulated as a drug, which would add both time and money to get to market and price.

Contact lens solutions that first entered the market contained thimerosal as a preservative and were well tolerated by almost everyone. With time, more and more people became allergic and had to discontinue use of thimerosal-containing solutions. This is because thimerosal acts like a hapten. Haptens are small molecules that bind to larger compounds such as proteins. Continued exposure to the bound compounds (hapten-adduct) leads to sensitization. Binding of these compounds can occur with several types of proteins, even those produced naturally by the body. So, haptens, in theory, could be a contributing factor in development of some autoimmune diseases.

Some trace minerals in the contrast materials used by radiologists (e.g. iodine) can also cause allergic reactions. Iodine (as opposed to iodide used in table salt) binds to proteins to form iodoproteins, which can cause sensitization in a mechanism similar to that of hapten-adducts. Once the body is re-exposed to the compound, an antigen-antibody reaction can ensue and may cause anaphylaxis. Thus, it is reasonable to believe that addition of iodine to wound care products will follow the same course as bacitracin and thimerosal, and the rates of sensitivity will increase over time. In wound care products, iodine should be substituted for safer alternatives.

In summary, modern wound care products provide a protective barrier from further irritation and insult and optimize moisturization of the wound. Some have added microbiocides and antimicrobials with unavoidable, innate toxicity that result in more and more people becoming allergic. These problems could be minimized by the exclusive use of non-allergic food ingredients. At this writing, however, few if any wound care product are made entirely of foods.

II Safe and Effective Food-Based Wound Dressings have not been Commercially Available Because of the Difficulty of Obtaining Standardized Ingredients and Conforming to Good Manufacturing Practice Regulations (GMP)

The recognized “Father of Western Medicine”, Hippocrates, advised physicians to: “First do no harm” (Epidemics, Bk. I, Sect. XI). He also is credited with teaching, “Let your food be your medicine and your medicine your food.” The “Father of Modern Pharmacology and Toxicology”, Paracelsus, wrote “The art of healing comes from nature, not from the physician. Therefore, the physician must start from nature with an open mind.”

In well-controlled clinical trials, foods such as potato peel, banana leaf and honey have proven to be equal to or better than current standard-of-care for burns and wounds. However, honey, potato peel and banana leaf dressings haven't been able to obtain regulatory approval for marketing because they cannot be manufactured and packaged under international good manufacturing practice (GMPs) regulations. Therefore, though more safe and effective than current standard of care, they can't be made available in a consistent format to retail outlets, hospitals or health care providers. This invention will provide the safe benefits of natural food-based wound dressings that are manufactured under GMPs and can be sold in a consistent format in retail settings. Products made under GMPs must include standardized ingredients that, at a minimum, meet the requirements of the FCC (Food Chemicals Codex).

A study conducted by the Department of Surgery, LTM General Hospital and LTM Medical College, Mumbai, India, compared banana leaf dressing (BLD) to vaseline gauze (VG) dressing, the current standard of care for donor graft sites. BLD and VG were compared in their ability to relieve pain, promote early epithelialization of donor areas, and lessen pain in dressing removal. BLD performed better on every score than VG (P<0.001).⁴ Another study by the same hospital in India compared the efficacy of boiled potato peel bandage to banana leaf dressing in partial thickness burns. Potato peel bandages had been used in this burn unit for nine years prior to this study. The study concluded that both banana leaf and potato peel dressings had equal efficacy in protecting the wounds and aiding healing.⁵

The University of Limpopo in South Africa compared honey to IntraSite Gel as wound healing agents. The healing times of shallow wounds, side effects and patient satisfaction were compared. In all parameters tested, the study found “no evidence of a real difference between honey and IntraSite Gel as healing agents”.⁶

A study comparing honey to silver sulfadiazine gauze dressing in management of superficial burn injuries found honey to be the superior dressing in control of infection, time to formation of healthy granulation tissue, healing time, pain relief, and incidence of scarring.⁷ Another study compared healing of abscess wounds with either honey or chlorinated lime and boric acid solution (EUSOL), a standard of care in many parts of the world. Honey-treated wounds demonstrated quicker healing and a significantly shorter hospital stay vs. those treated with EUSOL (t=2.45, p=0.019). The investigators concluded that honey is a superior wound dressing to EUSOL and is recommended for dressing infected wounds.⁸

### III Preservatives in Current Standard-of-Care Wound Care Products Cause Harm to Healthy Immune System Cells and Delay Wound Healing

Currently, most wound treatment is directed at controlling infection. Many of these treatments are considered too toxic to be taken orally. For example, Dakin's solution is composed of bleach and boric acid. Hydrogen peroxide, iodine-containing products, scarlet red, Silvadene, Mercurichrome, Panafil, and topical antibiotics are too toxic for internal use. It is falsely assumed that these ingredients are not absorbed systemically or do not cause systemic problems. These biocides are intended to kill bacteria in a wound and, thus, prevent infection; however, the effect they also have on the delicate cells required for the healthy wound healing process has not been evaluated.

Though hydrogels are currently the safest form of wound dressing, most are preserved from microbial contamination by the use of synthesized preservatives. Because of the innate toxicity of these preservatives, their allowable limits in food are very low. Most of these are acids, such as sodium benzoate, sodium metabisulfite, potassium sorbate, calcium ascorbate, calcium propionate, calcium sorbate, potassium bisulfite, potassium metabisulfite, sodium ascorbate, sodium bisulfite, sodium propionate, sodium sorbate, sodium sulfite. Another example is methylparaben, the safety of which has recently come into question. All these food-grade preservatives are biocides that, by nature, kill or prevent the growth of bacteria. However, not only do they damage microbes, but they also can cause harm to healthy cells. When taken internally in foods, these preservatives become mixed with other components in the GI tract and are thus rapidly diluted so as not to cause harm. However, when used in topical wound dressings, they remain in contact with the wound and with the immune cells for an extended period of time. In essence, these preservatives can actually damage immune cells and slow the wound healing process. It is likely that the absence of preservatives in the honey, potato peel and banana leaf dressings enabled them to outperform the commercially available dressings.

IV Systemic and Locally-Applied Analgesics for Pain Relief in Wounds have Adverse Side Effects Such as Drowsiness, Constipation, and Dependency

Topical anesthetic drugs in current use, such as lidocaine, tetracaine, benzocaine, and prilocalne, work by blocking primarily sodium ion (Na) channels so the sensation of pain cannot be transmitted. Concerning topical anesthetic drugs, the U.S. Food and Drug Administration (FDA) has warned, “Adverse events consistent with high systemic exposure to these products include seizures and cardiac arrhythmias”.^{9,10} This patent describes a wound care product that stops pain by the mechanical (non-drug) process of trapping ions in the dressing at the wound site (like flies on flypaper) before the ions can be mobilized to initiate and transmit the pain. The presence of ions, such as sodium and calcium, are responsible for pain signaling to the brain. A dressing applied to an open wound comes in direct contact, not only with delicate living cells required for wound healing, but also with nerves that transmit pain signals to the brain. Ions such as Na and calcium (Ca) are required for pain signaling.

Pain is the interpretation and expression by the brain of sensory input from nociceptive neurons and environmental stimuli.¹¹ The nociceptor is the peripheral end of a primary afferent nociceptive neuron that responds to stimuli that threaten or actually damage tissue.¹² There are nociceptors throughout the body surface, and also in the muscles, joints and viscera.¹² Nociceptors are activated by many different stimuli that lead to the alteration of ion concentrations, most significantly, Na, Ca, and potassium (K), across the nociceptor and neuronal membrane.¹³ The physiological role of Na, Ca, and K, the channels that allow them entry into or out of the neuron, and the role of pharmacological agents affecting these ions will be discussed here. The environmental and psychological aspects of pain are not discussed here.

### Initiation of Nociceptive Impulses

In contrast to connecting axonal regions of neurons, the nociceptive terminal axons look like a chain of beads.¹³ These beads contain increased amounts of mitochondria and vesicles, and they are uncovered or only partially covered by Schwann cells.¹³ This lack of a Schwann cell covering of the receptor allows for better access of noxious stimuli to the receptor membrane.¹³ In the nociceptive terminal axon, there are two repeating regions, the generator regions and the regenerator regions. The generator regions are the beads, and the regenerator regions are the area between the beads.¹³ There are several gated ion channels in the generator region that respond to noxious stimuli, such as the TRPV-1 receptor (discussed below). Upon activation of these gated ion channels, Na and Ca enter the neuron, and the generator region produces generator or receptor potentials which are potentials graded in proportion to how many Na and Ca ions enter the generator region.¹³ The regenerator region is the site where initiation of propagating impulses occurs and contain a high concentration of tetrodotoxin—resistant type voltage-gated Na channels (discussed below).¹³ The regenerator region is also the site where pharmacologic action occurs.¹³ A series of regenerative potentials from the chain of generator and regenerator regions could interact with each other determining what is sent by the individual nociceptive neuron to the CNS.¹³

### Na (Role of Ion, Channels, Drugs)

Na is primarily responsible for the depolarization of the neuron.¹³ Na concentrations across the neuronal membrane are maintained at a ratio of 10:1 extracellularly to intracellularly by the Na/K ATPase (Blankenship, 2003). Na then enters the neuron upon activation of various Na ion channels such as Tetrodotoxin—Resistant and Tetrodotoxin—Sensitive voltage-gated Na ion channel¹²⁻¹⁵ and TRPV-1 ion channels¹³ to cause depolarization of the neuron, this is discussed further below.

An example of Na influx at the nociceptive terminal is the TRPV-1 receptor. This type of receptor is a gated receptor and is localized primarily in the generator region of nociceptive receptor membranes.¹³ The TRPV-1 receptor has multiple roles, and is responsible for the influx of both Na and Ca into the cell. The effects of the TRPV-1 channel with respect to the Na ion will be discussed here, and the effects of the TRPV-1 channel with respect to the Ca ion will be discussed under the section on the Ca ion.

The TRPV-1 receptor is a temperature-gated ion channel that responds to capsaicin, noxious heat, hydrogen ions, and noxious chemical stimuli.¹³ The TRPV-1 receptor is located primarily on the nociceptive neuron terminal as opposed to its axonal trunk or soma.¹³ Upon activation of the TRPV-1 receptor, the channel opens and Na ions (and Ca ions) enter the neuron¹³ down their concentration gradient leading to depolarization¹⁴ in the generator regions of the nociceptive terminal.¹³ As Na enters the generator region of the nociceptive neuron terminal, the generator region is depolarized toward threshold.¹³ If enough Na channels at the generator region of the nociceptive neuron receptor are activated to where enough Na ions enter the nociceptive generator regions to yield a depolarization great enough, the voltage-gated Na channels are activated in the regenerator region.¹³ The action potential is then propagated down the axon to the central nervous system by the Tetrodotoxin-sensitive voltage-gated Na channels.¹³

The influx of Na into the neuron through voltage-gated Na channels causes the rising phase of the action potential.¹³ The structure of voltage-gated Na channels is influenced by the membrane potential.¹⁴ Voltage-gated Na channels have two different gates, the activation gate and the inactivation gate, that respond inversely to each other with depolarization of the membrane.¹¹ The channel itself has three phases.¹¹ In the resting or closed phase, the activation gate is closed and the inactivation gate is open.¹¹ When a depolarizing membrane potential approaches the voltage-gated Na channel, a structural change occurs,¹³ and the activation gate opens rapidly allowing Na to enter the cell.¹¹ Shortly after the activation gates open, another structural change occurs and the inactivation gates close.¹¹ Once repolarization of the membrane occurs, the activation gate closes first, then the inactivation gate opens as the channel returns to its resting phase.¹¹ The nociceptive neuron has many voltage-gated Na channels that can be seen as being in series which allows for the propagation of the action potential in the nociceptive neuron.^{11,13-16}

Na flux across the neuronal membrane is targeted by various pharmacological agents such as local anesthetics, class I antiarrythmics, and some antiepileptic drugs.¹³ Local anesthetics such as cocaine, lidocaine, bupivacaine, and procaine, cause a reversible block of the conduction of action potentials down the neuron.¹⁷ Local anesthetics act primarily at the cell membrane by preventing the influx of Na by binding to sites within voltage-gated Na channels.¹⁷ Conduction down the neuron ceases due to: 1) a decrease in the action potential propagation down the neuron, 2) an increase in the electrical excitability threshold, 3) a decrease in the rate of rise of the action potential, and 4) subsequent slowing of the conduction impulse.¹⁷

As stated above, local anesthetics act on voltage-gated Na channels. Therefore, any voltage-gated Na channel that the local anesthetic has access to will be affected by the local anesthetic. The different physiologic properties and distribution of voltage-gated Na channels in different nerve fibers leads to an order of disappearance of function with anesthetic use. First, voltage-gated Na channels of pain fibers are inactivated, then voltage-gated Na channels of neuron fibers for temperature, touch, deep pressure, and, finally, motor function are inactivated.¹⁷ From another perspective, C fibers are blocked, then small myelinated Adelta fibers are blocked. Then, with increasing concentration of local anesthetic, the larger myelinated Agamma (Aγ), Abeta (Aβ), and Aalpha (Aα) fibers are blocked.¹⁷ Local anesthetics can have adverse effects on the CNS and other systems with conduction.¹⁷ CNS depression can lead to death by respiratory failure. Rarely, in the cardiovascular system, ventricular tachycardia or ventricular fibrillation can occur, except with bupivacaine where these cardiovascular side effects are more common.¹⁷ Other systems are also affected and can be reviewed in Hardman, 2001.

Vasoconstrictors such as epinephrine can prolong the action of local anesthetics by decreasing the rate of absorption of the local anesthetic¹⁷. This is accomplished by decreasing the blood flow in tissue¹⁷. This also keeps the local anesthetic in the target area.¹⁷ On the other hand, vasoconstrictors can cause delayed wound healing, tissue edema and necrosis due to increased oxygen demand and decreased supply.¹⁷ Vasoconstrictors should not be used in areas with limited collateral circulation due to lack of oxygen supply with excessive vasoconstriction and increased local metabolism.¹⁷ The expanding knowledge of TRPV-1, 2, and 3 receptors have opened up new possibilities for analgesics that act selectively on nociceptors.¹³

### Na Channels and Pathologic States

Voltage-gated Na channel expression changes with different types of peripheral nerve damage; tetrodotoxin-resistant type voltage-gated Na channels are down regulated and tetrodotoxin-sensitive type voltage-gated Na channels are up regulated.¹³ In damaged dorsal root ganglion neurons, there appears to be an increase in the current of tetrodotoxin—sensitive voltage-gated Na channels associated with hyperexcitability of the neuron.¹³ Clinical evidence for voltage-gated Na channel activation in neuropathic pain is supported by the observation that Na channel blockers, like the local anesthetic lidocaine, are effective in reducing spontaneous pain in hyperalgesia and allodynia in different neuropathic diseases.¹³ Examples of genetic and physiologic evidence of the alteration of voltage-gated Na channel expression can be found in Oh, 2006.

### Ca (Role of Ion, Channels, Drugs)

The increase of intracellular Ca concentration increases the response of the nociceptive membrane to excitation.¹³ The Ca ion plays a major role in signal transduction which is involved in regulation of neurotransmitter release.¹³ Experiments showed that different types of Ca channels are involved in release of pain-related neurotransmitters.¹³ These neurotransmitters, namely glutamate, Substance P, and calcitonin gene-related peptide, have been shown to contribute to the sensitization of spinal processing of pain signals centrally,¹³ and these neurotransmitters have been shown to contribute to peripheral sensitization of the nociceptive terminal.¹² Increases in intracellular Ca ion concentration also play a role in membrane excitability, electrical spiking behavior, gene expression, and pain perception.¹³

Ca ions enter the cell down their concentration gradient when channels are opened on the nociceptive neuronal membranes that are permeable to the Ca ion.¹³ As with the Na ion, there are basically two broad classes of Ca channels—the voltage-dependent Ca channels and channels that respond to other stimuli, namely noxious temperature and noxious chemicals such as the TRPV-1 receptor.¹³

With respect to the Ca ion, once activated, the TRPV-1 receptor allows influx of the Ca ion.¹³ This increase in intracellular Ca ion concentration is the determining step in sensitization of the nociceptor.¹² The increase in intra-neuronal Ca ion concentration leads to alteration of responsiveness of sensory neurons to stimuli by the activation of second messenger cascades such as protein kinase C, protein kinase A and protein kinase G pathways.¹² These second messenger cascades act on ion channels and on membrane receptors such as TRPV-1.¹² An example given in Willis and Coggeshall shows that activation of certain membrane receptors by a diffusible second messenger results in an increase in voltage-gated currents through tetrodotoxin-resistant Na channels.

Voltage-dependent Ca channels have similar evolutionary origin to Na and K channels (Blankenship, 2003). There are several types of voltage-dependent Ca channels involved in the release of neurotransmitters related to pain: L-type, N-type, P/Q-type, R-type, and T-type¹³. These five types of voltage dependent Ca channels are divided into 2 classes by the membrane potential at which they are activated; high voltage versus low voltage¹³. High voltage-dependent channels are L-type, N-type, P/Q-type, and R-type¹³. Low voltage-dependent channels are T-type¹³. These channels are distinguished by voltage dependence, kinetics, and pharmacology¹³.

L-type voltage-dependent Ca channels are involved in nociception in dorsal root ganglion cells and in the spinal cord.¹³ They are involved in the release of substance p. 13 Nifedipine is an L-type specific voltage-dependent Ca channel blocker and will block the release of Substance P which is usually released by mediators of pain and inflammation.¹³ In clinical application, conflicting results have been obtained for pain modulation with respect to location and modality of administration of L-type specific Ca channel blockers.¹³

N-type voltage-dependent Ca channels are involved in nociception by mediating synaptic transmission in the CNS.¹³ They are also involved in release of neurotransmitters associated with pain signaling: glutamate, Substance P and calcitonin gene-related peptide.¹³ There is clinical evidence that N-type Ca channels can be targeted for analgesic therapy for neuropathic and inflammatory pain, but not for acute pain.¹³ N-type Ca channel blockers have adverse effects in a dose dependent manor.¹³ In genetic studies, N-type Ca channel knockout mice have decreased allodynia and hyperalgesia.¹³ A newer class of potential analgesics known as conotoxins is derived from the venom of marine cone snails, and some components of these conotoxins target N-type voltage-dependent Ca channels (Snutch, 2005).

P/Q-type voltage-dependent Ca channels have a role in the release of neurotransmitters associated with pain in the CNS like the N-type Ca channels.¹³ The neurotransmitters associated with P/Q-type Ca channels are glutamate, serotonin, norepinephrine, Gamma(γ)-Amino Butyric Acid (GABA), and glycine. The role of P/Q-type Ca channels with respect to pain may be at the spinal level; however, their exact role has been difficult to elucidate due to low survival in genetic knockout studies.¹³

R-type voltage-dependent Ca channels may have a role in the periaqueductal gray in reducing the behavioral response to pain.¹³

T-type voltage-dependent Ca channels are low voltage-dependent Ca channels.¹³ These channels are active in acute pain, and may work by a pronociceptive mechanism by boosting the pain signal centrally and peripherally.¹³ T-type Ca channels work by signal suppression in the thalamus with persistent pain signals.¹³ In contrast, in neuropathic pain, T-type Ca channels lower threshold and promote bursting activity, thus inducing peripheral hyperexcitability.¹³ T-type Ca channels are involved in the induction of long-term potentiation at synapses in the central nervous system by alterations of the plasticity of these synapses.¹³

Influx of Ca ions results in release of sensory neuropeptides, including calcitonin gene-related peptide, Substance P and many others (Oh, 2006, Willis and Coggeshall, 2004). This release is both central and peripheral.¹² Peripheral release of neuropeptides plays a role in neurogenic inflammation.¹² Substance P causes plasma extravasation, and calcitonin gene-related peptide causes vasodilation,¹² Substance P has been shown to play a role in sensitizing nociceptor terminals by increasing the effect of inflammatory mediators.¹²

Some of the evidence that pharmacological agents targeting voltage-dependent have a role in analgesia has been discussed above. There have been studies done with voltage-dependent Ca channel blockers studying their effect on pain. Studies support the use of voltage-dependent Ca channels blockers on pain. These provide analgesia when administered.¹³ The current problem with Ca channel blockers and their use for analgesia is that they lack selectivity and specificity to various Ca channels.¹³ The response to blocking of Ca channels depends on the specific drug, dosage and route of administration.¹³ For Ca channel blockers to be used for analgesia, more effective and selective drugs are needed.¹³ Currently, gene knockout technologies are being used to identify the role of various Ca channels in pain.¹³

One mechanism for opioid use in pain is suppression of voltage-gated Ca currents.¹⁷ This suppression blocks neurotransmitter release and the transmission of pain in various pathways.¹⁷ This mechanism may be coupled to various second messengers like MAP kinases and the Phospholipase C cascade.¹⁷

### K (Role of Ion, Channels, Drugs)

The K ion determines the resting membrane potential.¹⁴ This is due to the fact that resting membrane is permeable to K ions and virtually impermeable to other ions.¹⁴ Nociceptors express transient voltage-gated Kv 1.4 channels^{12,13} which undergo rapid N-type inactivation. Activation of these voltage-gated K channels leads to decreased excitability of the nociceptive neurons, and inhibition of these voltage-gated K channels leads to hyperexcitability of the nociceptive neurons.^{12,13} In ligated spinal nerves, there is a reduction in Kv 1.4 type K channels, and this could be partially responsible for the hyperexcitability of the nociceptors.^{12,13} The Kv 1 family of channels may be potential targets for pharmacologic action in preventing neuropathic pain by increasing the duration or enhancing the activity of the Kv 1.4 channel.¹³

Local anesthetics interfere with K channel function in addition to Na channel function.¹⁷ A higher concentration of local anesthetics is required to affect K channels compared to Na channels.¹⁷ Since the nociceptive neuron is permeable to K and there is a higher concentration of local anesthetic required to affect K channels, there is not a large or consistent change in resting membrane potential.¹⁷

### Conclusion:

Na, Ca, and K ions ultimately control the fate of the nociceptors. The various events these ions cause or prevent and the channels that allow their passage have been targets of analgesic agents.

### Current Wound Care Products can Cause Excessive Inflammation

The increase of intracellular Ca concentration increases the response of the nociceptive membrane to excitation. The Ca ion plays a major role in signal transduction which is involved in regulation of neurotransmitter release. Experiments have shown different types of Ca channels are involved in the release of pain-related neurotransmitters. These neurotransmitters, namely glutamate, Substance P, and calcitonin gene-related peptide, have been shown to contribute to the sensitization of spinal processing of pain signals centrally, and also these neurotransmitters have been shown to contribute to peripheral sensitization of the nociceptive terminal. Increases in intracellular Ca ion concentration also play a role in membrane excitability, electrical spiking behavior, gene expression, and pain perception.

Influx of Ca ions results in release of sensory neuropeptides including calcitonin gene-related peptide, Substance P and many others, both centrally and peripherally. Peripheral release of neuropeptides plays a role in neurogenic inflammation. Substance P causes plasma extravasation, and calcitonin gene-related peptide causes vasodilation. Substance P has been shown to play a role in sensitizing nociceptor terminals by increasing the effect of inflammatory mediators. All these second messengers require Ca, and by binding Ca we are able to reduce excessive, prolonged and painful inflammation.

### SUMMARY

A rationally designed wound care product and its development, manufacture and uses, particularly hydrogel wound dressings, that are made entirely of naturally occurring food ingredients, and optionally with safe food additives.

One aspect of this invention is to provide the safe benefits of natural food-based wound dressings that are standardized and manufactured under GMPs and can be sold in retail settings.

Another aspect of this invention provides a safe wound care product that will not harm healthy immune cells or delay the healing process and that breaks down into nutrients that are useful to the body, rather than into drugs that can be harmful.

Yet another aspect of this invention will provide a hydrogel with enhanced Ca-, Na-, and K-binding capability that stops pain by actually trapping ions (like flies on flypaper) so that the pain signal cannot initiate or transmit down these channels.

One way this invention advances each of the characteristics of wound products is discussed below.

1. Are Composed of Food Ingredients:

This patent teaches the art of formulating wound care products that are composed entirely of ingredients regulated as foods.

Are safe and effective (i.e. does no harm)

“Antigens absorbed through the gut are first ‘seen’ by liver macrophages, which remove immunogenic aggregates, etc., leaving only soluble ‘tolerogen’.”²⁰ A tolerogen is an antigen that induces a state of specific immunological unresponsiveness to subsequent challenging doses of the antigen. (Dorland's Medical Dictionary for Health Consumers.© 2007 by Saunders, an imprint of Elsevier, Inc. All rights reserved) “In addition, antigen-presenting cells in the gut may be specialized for tolerance induction, to prevent immune responses against food. Trials are in progress to see whether administering self antigens (such as collagen for rheumatoid arthritis, or myelin basic protein for multiple sclerosis) can be used in treatment of autoimmunity.²⁰ This “tolerogen principle” means that ingredients consumed as foods are not normally expected to create allergenic responses when applied topically. This patent teaches the art of making safe and effective wound care products entirely of food ingredients.

Have an osmotic pressure that is compatible with optimal healing:

Most of the approximate 400 products on the market are designed to be applied to wounds with specific moisture content—light, medium or heavy. Products for oozing wounds are designed to absorb moisture (>280 mosm); products for normal wounds are designed to maintain moisture (100-280 mosm); products for dry wounds are designed to contribute moisture (<280 mosm). This patent teaches the art of making all three types of dressings entirely of food ingredients

4. Are Buffered so as to Maintain the Optimal pH Throughout the Healing Process:

Currently, available wound dressings to our knowledge are not buffered, but are only pH-adjusted to a physiologically acceptable level, so they cannot maintain the optimal pH throughout the healing cycle. The patent teaches the art of choosing food ingredient combinations that can provide buffering to maintain the dressing at a pH slightly less than that of blood (the optimal pH for wound healing) until the wound is healed. For example, monobasic ammonium phosphate and dibasic ammonium phosphate are both available in Food Chemical Codex (FCC) grade. These two ingredients can be combined to make an excellent buffer that maintains the pH of the dressing at a range of 7.0 to 7.4, the preferred range: 7.25 is optimal. A workable range is pH of 6.0-8.0. The pH should never be lower than 4.0 nor greater than 11.0.

5. Provide a Protective Barrier from Further Irritation and Insult to the Wound:

All wound dressings are designed to provide barriers from further injury and irritation to the wound. This patent teaches the art of making such products entirely of safe food ingredients.

6. Control Bacteria Found in the Skin and Mucosa:

Currently, antiseptics and antibiotics are used to control bacteria in wounds, but bacteria can become resistant to these chemicals. However, all plants naturally contain biochemicals that control bacteria; if they didn't, bacterial attack would soon render them extinct. Bacteria don't become resistant to these natural biochemical “preservatives”. In this patent we teach the art of using biochemical preservatives found naturally in plants to control bacteria in wounds, rather than synthetic chemicals that cause harm.

7. Control Both Bacteria and Viruses Found in the Skin and Mucosa:

In addition to biochemicals that control bacteria, plant foods also naturally contain biochemicals that control viruses. This patent teaches the art of using natural food ingredients that have antibacterial and antiviral properties.

8. Control Bacteria, Viruses and Fungi Found in the Skin and Mucosa:

In addition to biochemicals that control bacteria and viruses, plant foods also naturally contain biochemicals that control fungi. This patent teaches the art of using natural food ingredients that have antibacterial, antiviral and antifungal properties.

9. Nourish Wounds:

Many wound care products absorbed by the wound may in fact cause harm as described above. This patent teaches the art of creating a wound dressing that nourishes the delicate cells involved in wound healing right at the wound site. Polysaccharides, phosphates, amino acids and citric acid cycle intermediates can actually do this; for example, macrophages are attracted to polymannans, which they “grab” with their mannose receptors, phagocytize, and break down into mannose. The mannose is then used by the macrophage as energy and raw material to produce monokines at a faster rate. Thus, the macrophage, which orchestrates wound healing, can accomplish its task faster and more optimally.

10. Control Excessive, Prolonged Inflammation:

As discussed in the section on inflammation above, second messengers require Ca, and by binding Ca we are able to reduce excessive, prolonged and painful inflammation. Many food products contain polysaccharides, phosphates, amino acids and citric acid cycle intermediates, which can bind excess calcium. The inflammatory process, which is necessary for healthy wound healing, lasts no longer than is necessary for optimal wound healing. This patent teaches how to prepare wound products containing safe food ingredients that control excessive inflammation and thus help minimize scarring.

11. Minimize Allergenic Potential:

As described in number 2 above, many wound care products on the market have the potential to sensitize the patient and create allergic reactions or even anaphylaxis. This patent teaches the use of safe food ingredients in wound care to create ‘tolerogens’ instead of antigens.

12. Minimize Irritation Potential:

Many of the synthetic ingredients, antimicrobials, antiseptics, preservatives, and other chemicals added to wound care products can cause irritation. This patent teaches the art of using safe food ingredients instead of irritating chemicals as ingredients in wound care products.

13. Are Easy to Use or Apply:

Many current wound care products are difficult to apply and can be difficult to remove from the wound site, which can lead to further damage of the healing tissue. This patent teaches the art of using food ingredients that are water soluble and thus are easy to apply and easy to remove. 14. Pass the preservative challenge test required for products intended for multiple use:

Currently there are no multiuse wound products made entirely of food ingredients that can be legally marketed. To be legally marketed, the product must pass the preservative challenge test (USP <51>). The test, including criteria for passing the test, are described in the U.S. Pharmacopeia. This patent teaches the art of using natural food ingredients to create a product that will pass the preservative challenge test.

15. Control Pain Without the Adverse Effects Associated with Analgesic Drugs:

The present invention teaches how to choose ingredients with enhanced Na- and Ca-binding capacity. A wound dressing made with such ingredients traps ions responsible for pain signaling (like flies on flypaper), thus preventing initiation and transmission of the pain signal. This wound dressing can also be safely used in combination with systemic and local anesthetics with no additional adverse effects. Examples of Na- and Ca-binding ingredients are polysaccharides, dicarboxylic acids, amino acids, phosphates and monosaccharides, such as xylitol. The pain-reducing ability of the dressing can be measured using the art taught in a new provisional patent application by the above inventors for METHOD FOR MEASURING IONIC SEQUESTERING POTENTIAL OF HYDROGELS filed Apr. 26, 2007, Ser. No. 60/926,396.

16. Can be Individually Optimized Based on the Diet of an Individual or a Group of People:

This patent teaches the art of customizing safe wound dressings for the individual or group based on their dietary habits. This practice would further minimize potential allergies caused by wound products. For example, based on the “tolerogen principle,” meat-based ingredients should not be used in wound dressings for vegetarians, and alginate dressings are more likely to cause allergic reactions in groups who don't consume algaes in their diets.

17. Are Composed of Standardized Food Ingredients that can be Produced for the Mass Market Using Good Manufacturing Practice Guidelines:

An aspect of the present invention teaches the art of making wound products with FCC-grade ingredients or better that can be manufactured under GMPs and marketed internationally. Sigma-Aldrich, Spectrum and other similar companies can supply FCC grade products with certificates of analysis required for GMPs.

18. Contain fragrant essential oils and other food ingredients to take advantage of the benefits provided by aromatherapy, such as calming effects and pain control. These oils (lavender, orange, cedar, jasmine, peppermint, rosemary, sage, and sandalwood) produce a calming effect, mainly by increasing the levels of GABA in the brain.

Another aspect of the present invention teaches the advantage of adding essential oils to wound dressings. These provide the benefits of aromatherapy, which have been shown to lessen pain and have a claming effect. Fragrant oils also mask unpleasant odors sometimes associated with wounds.

### ADVANTAGES

Yet, another aspect of the present application provides a rationale and a design method for making a new field of wound care products entirely out of food ingredients. These products can be standardized, manufactured under good manufacturing practice guidelines (GMPs) and made available to the mass market. They can also be personalized to the diets of individuals or groups to minimize allergenic responses. The advantages provided by the new art described in this patent are listed above in “Summary of the Invention.” A few examples of advantages are: lack of adverse effects like those associated with drugs and support for natural healthy wound healing by provision of topical nutrition for the delicate cells involved in that process.

### How it Solves Problems

Still another aspect of the present invention solves problems in the ways described above in “Summary of the Invention”. One aspect of the present invention teaches the art of producing a broad class of wound care products for management of all wounds and associated pain using only biochemicals naturally found in food.

For example, instead of using local (lidocaine) or systemic (vicodin) drugs to control pain (with the adverse effects associated with their use, e.g. dependency, drowsiness, constipation etc.), this invention also teaches the art of using only safe foods to control pain. Also, this patent teaches a method for controlling bacteria, viruses, and fungi with food ingredients rather than with drugs or other chemicals that do not occur naturally in food.

### STATEMENT OF THE OBJECT OF THE INVENTION

One object of the present invention is to provide a rational design for a way to make a new class of wound care products made entirely of food ingredients that can be adjusted to have up to 18 of the characteristics listed in “Field of Endeavor” discussed later.

### DETAILED DESCRIPTION OF PREFERRED EMBODIMENTS

Generally, this invention will provides a wound care product with enhanced Ca- Na- and -K binding capability for use in the treatment and management of wounds. The product stops pain by the mechanical (non-drug) process of trapping ions in the dressing at the wound site (like flies on flypaper) before the ions can be mobilized to initiate and transmit pain. One aspect of this invention provides a wound care product that helps control excessive, prolonged and painful inflammation. Minimization of inflammation will also minimize scarring. Moreover, in contrast to topical anesthetic drugs, this dressing breaks down into nutrients that are useful to the body, rather than into drugs that can be harmful.

### Definitions

As used herein, “food” refers to a material consisting essentially of protein, peptide, amino acid, carbohydrate, essential oil, and fat of plant, animal or microbial origin used by the body of an organism to sustain growth, repair, and vital processes and to furnish energy.

As used herein, “food additive” refers to substances which may, by their intended uses, become components of food, either directly or indirectly, or which may otherwise affect the characteristics of the food. The term specifically includes any substance intended for use in producing, manufacturing, packing, processing, preparing, treating, packaging, transporting, or holding the food, and any source of radiation intended for any such use.

Weight percent is calculated by dividing the weight of a reagent by the total weight of a mixture to which it is added subsequent to the addition of the reagent. For example, adding 1 gram of a reagent A to 99 grams of a reagent B, thereby forming 100 grams of a mixture A+B would constitute adding 1 weight % of the reagent A to the mixture.

By “effective amount” is meant that amount which will provide the desired beneficial wound-care effect or response in a mammal. For example, the effective amount varies from one food ingredient to the other; also, it varies from mammal to mammal. It should be understood that effective amounts of food ingredients or food additives will vary. Thus, while one mammal may require a particular profile of food ingredients, food additives, or both present in defined amounts, another mammal may require the same particular profile of food ingredients, food additives, or both present in different defined amounts. Effective amount also means that amount that is sufficient to cause the product to pass the preservative challenge test described in the U.S. Pharmacopeia USP <51>, the entire content of which is incorporated herein by reference, and to obtain market approval from governmental regulating agencies.

By “wound” is meant any type of injury to a body, including physical burns, chemical burns, chapped lips, partial thickness skin grafts, full thickness skin grafts, skin flaps, biopsy sites, excision biopsy sites, punch biopsy sites, shave biopsy sites, fine needle aspiration sites, suture sites, suture removal sites, staple sites, staple removal sites, wounds closed with adhesive compounds, wounds closed with adhesive strips, wounds closed by secondary intention, tattoos, areas treated with lasers, areas treated with Intense Pulsed Light, areas treated with chemical peals, areas treated with dermabrasion, areas treated with micro-dermabrasion, areas of hair transplants, dermatitis, intravenous catheter sites, cutaneous penetration site of drains including Jackson-Pratt and Penrose, cuntaneous penetration site of chest tubes, injection sites, immunization sites, insulin injection sites, intramuscular injection sites, sites of local anesthetic administration, sites of injection of anticoagulants, sites of injection of cosmetic paralytics including BOTOX® (botulinum toxin), sites of injection of cosmetic filling and contouring agents including Restylane® (hyaluronic acid), Sculptra™(poly-L-lactic acid) and Radiesse™ (calcium hydroxylapatite), sclerotherapy injections, mesotherapy injections, cutaneous penetration site of all transcutaneous internal fixation devices, cutaneous penetration site of all transcutaneous external fixation devices, wounds obtained during piercing, diabetic ulcers, foot ulcers, pressure ulcers (stage 1-4), skin conditions associated with peristomal care, radiation dermatitis, sunburn, cuts, abrasions, or any combination of these conditions.

### Field of Endeavor

The present invention relates to a rational design method for developing wound care products that:

1. are composed entirely of food ingredients,

2. are safe and effective,

3. have an osmotic pressure that is compatible with optimal healing,

4. are buffered so as to maintain the optimal pH throughout the healing process,

5. provide a protective barrier from further irritation and insult to the wound,

6. control bacteria found in the skin and mucosa,

7. control both bacteria and viruses found in the skin and mucosa,

8. control bacteria, viruses and fungi found in the skin and mucosa,

9. nourish wounds,

10. control excessive, prolonged inflammation, and thus minimize scarring,

11. minimize allergenic potential,

12. minimize irritation potential,

13. are easy to use or apply,

14. pass the preservative challenge test required for products intended for multiple use,

15. control pain without the adverse effects associated with analgesic drugs,

16. can be individually optimized based on the diet of an individual or a group of people,

17. are composed of standardized food ingredients that can be produced for the mass market using good manufacturing practice guidelines, and

18. contain fragrant essential oils and other food ingredients to take advantage of the benefits provided by aromatherapy.

Specific Problems with Conventional Technologies Related to the Current Invention

1. Are Composed Entirely of Food Ingredients:

Currently, few, if any, wound care products on the market are composed entirely of food ingredients.

2. Are Safe and Effective:

Currently, many products on the market damage the delicate cells involved in the wound healing process and slow healing.

3. Have an Osmotic Pressure that is Compatible with Optimal Healing:

Though approximately 400 marketed wound care products have osmotic pressure compatible with optimal wound healing, we know of none that are made entirely of food ingredients.

4. Are Buffered so as to Maintain the Optimal pH Throughout the Healing Process:

Wounds heal optimally at a pH slightly more acidic than blood. Currently available wound dressing, to our knowledge, are only pH adjusted to a physiologically acceptable level and, thus, cannot maintain the correct pH throughout the healing process.

6. Control Bacteria Found in the Skin and Mucosa:

Currently, there are no wound dressings on the market designed to control bacteria found in the skin and mucosa using only food ingredients.

7. Control Both Bacteria and Viruses Found in the Skin and Mucosa:

Currently, there are no wound care products made of food ingredients designed to be both anti-microbial and anti-viral. Recent research has shown that chronic non-healing wounds are the results of viral-bacterial synergistic pathogenesis.^{18,19}

8. Control Bacteria, Viruses and Fungi Found in the Skin and Mucosa:

Currently, there are no wound dressings on the market designed to control fungi found in the skin and mucosa using only food ingredients.

9. Nourish Wounds:

Currently, there are no wound dressings on the market designed to nourish wounds at the site of the wound. Products currently on the market are in fact toxic to the wound.

10. Control Excessive, Prolonged Inflammation:

Currently, there are no wound care products on the market designed to control excessive, prolonged inflammation. In fact, many products on the market can actually cause inflammation or aid in the inflammatory process. For example, Ca alginate is a common ingredient found in hydrogels¹, and Ca ions can promote the inflammatory process. For this reason many dermatologists and other health care professionals do not use hydrogels or other interactive dressings that manipulate the wound environment. Instead they select petrolatum and/or gauze.

11. Minimize Allergenic Potential:

Currently, wound products contain many non-food ingredients that the body can become allergic to such as antibiotics, analgesics, antiseptics, and drugs. Also, multiple-use wound products require the addition of preservatives which, with continued use, cause allergic reactions in many people. Some of these non-food ingredients are haptens, small molecules that cause sensitization and allergic reactions when bound to larger compounds like proteins. In fact, the recent addition of iodine to new wound care products will probably increase the rates of sensitivity over time, since iodine is a hapten. Currently, no marketed wound dressings are designed to minimize allergenic potential by using only food ingredients.

12. Minimize Irritation Potential:

Currently, there are no wound dressings on the market designed to minimize the potential for causing irritation by using only food ingredients.

13. Are Easy to Use or Apply:

Many products currently on the market are difficult to apply and remove from the wound site.

14. Pass the Preservative Challenge Test Required for Products Intended for Multiple Use:

Products designed for multiple applications must pass the preservative challenge test (USP 51). Currently, there are no wound hydrogels made entirely of food ingredients that pass the preservative challenge test (USP 51).

15. Control Pain Without the Adverse Effects Associated with Analgesic Drugs:

Pain is currently treated both at the central nervous system and in the peripheral nervous system. Opioids such as morphine are used in the central nervous system to treat pain. Opioids interact with receptors by mimicking naturally occurring opioid peptides known as endorphins. Opioids inhibit responses to painful stimuli, but they also have significant rewarding and addicting properties. Tolerance to opioids is also a downfall to their use, in that larger amounts must be used over time to provide the same level of analgesia. Non-steroidal anti-inflammatory drugs (NSAIDS) and acetaminophen have analgesic effects on both the central and peripheral nervous system. NSAIDS are used to treat milder pain and are more effective in pain where inflammation has caused sensitization of the pain receptor. Acetaminophen can also be used for mild pain but has no effect on the inflammatory component of pain. Local anesthetics are most commonly Na channel blockers and are injected locally. All of these methods for treating pain carry the risk of systemic toxicity. Finally, hydrogel wound dressings have been used to help assuage pain by creating a protective barrier between the wound and irritations from the external environment. Currently, there are no wound care products designed to control pain through enhanced coordinate covalent binding of Na and Ca.

16. Can be Individually Optimized Based on the Diet of an Individual or a Group of People:

No wound care product on the market is customized based on foods eaten by the individual or the population group (i.e. that are designed to take advantage of the “tolerogen concept” described below).

17. Are Composed of Standardized Food Ingredients that can be Produced for the Mass Market Using Good Manufacturing Practice Guidelines:

Currently, there are no wound gels composed of 100% food ingredients that can be manufactured under GMPs.

18. Contain fragrant essential oils and other food ingredients to take advantage of the benefits provided by aromatherapy, such as calming effects and pain control. These oils (lavender, orange, cedar, jasmine, peppermint, rosemary, sage, and sandalwood) produce a calming effect, mainly by increasing the levels of GABA in the brain.

Currently, few, if any, wound care products are designed to take advantage of the benefits provided by aromatherapy. Fragrances are currently added to cover up bad odors that are sometimes associated with wounds.

### Making the Hydrogel Wound Dressing—A List of Ingredients and Information on How to Choose and Adjust the Amounts of Each Ingredient

### Composition—Ingredients

### A. Hydrating Agent (Purified Water)

a. RO (Reverse Osmosis) DI (Deionized) water or distilled water.

b. The amount of hydrating agent added to the composition depends on the desired level of moisturization. The osmotic pressure of blood is 280 mosm. For wounds with the correct amount of moisture, a dressing of 280 mosm would maintain that pressure. For wounds that are too moist, the osmotic pressure should be greater than 280 mosm. For wounds that are too dry, the osmotic pressure should be less than 280 mosm. The approximate 400 wound care products on the market fall within these categories. Ingredient “A” is adjusted depending on which of the three products is desired. Special-needs products may require that the osmotic pressure be outside of these guidelines. The osmotic pressure can be measured using an osmometer.

c. The percent of for workable and preferred percent of the hydrating agent in any of the compositions depends on the type of wound the hydrogel wound dressing will be applied, thus establishing an appropriate osmolarity that is conclusive to maintaining optimal moisture at the wound site.

d. The preferred osmolarity for these compositions would be from 20 to 290 mOsm, more preferably from about 180 to about 220 mOsm and ideally around 80 mOsm, which allows the product to moisturize the wound.

### B. Gelling Agents

Any food-grade polysaccharide or gelatin is preferred. Where possible, gelling agents should be selected based on foods normally consumed in the diet of the individuals or group for whom the product is intended. Normally, the formula will contain one or more gelling agent, the total concentration of which will normally be between 0.5 and 5 percent by weight in the formula. Start with 0.5 percent, then 1 percent, then 2 percent, and adjust up or down depending on the desired texture of the dressing. A few products for special needs may contain more or less than 0.5 to 5 percent. (For example, wound powders can contain more than 90% gelling agent, especially those intended to absorb exudate. Thin dressings intended to coat the mouth to treat mucositis etc. may contain less than 0.5 percent gelling agent.) Viscosity guidelines can be obtained from the suppliers of the gelling agents. In dental products, only beta-bonded gelling agents are preferred.

Examples include:

Acacia gum (Gum Arabic); Agar; Alginic acid; Ammonium alginate; Carrageenan; Cellulose; Methyl cellulose; Hydroxypropyl cellulose; Hydroxypropyl methyl cellulose; Ethyl methyl cellulose; Carboxy methyl cellulose; Crosslinked sodium carboxy methyl cellulose; Enzymatically hydrolysed carboxy methyl cellulose; Gelatin; Gellen gum; Guar gum; Gum ghatti; Karaya gum; Konjac gum; Linze mushroom; Locust bean gum; Pectin; Processed eucheuma seaweed; Propane 1,2-diol alginate; Tara gum; Tragacanth; Undaria seaweed (75:1 concentrate); and Xanthan gum.

### C. Cross-Linking and Cation-Binding Agents

Cross-linking agents help hold the gel together and thereby enhance the viscosity of wound dressings. Cation-binding agents help bind (sequester) cations to aid in pain relief. Some agents can perform both functions (i.e. both hold the gel together and bind cations). Both types of agents should be used according to good manufacturing practice guidelines (GMPs). Depending on the clinical effect desired, optimal viscosity can range from that of water to that of a sheet of dried gel. The effectiveness has to be determined clinically based on the clinical effect desired. The cation-binding assay described herein can be used to measure the potential analgesic and anti-inflammatory properties of the dressing. The percentages of these components should not exceed allowable limits in food for each country in which the producted is marketed.

a. Emulsifiers

To solubilize oil and water components and increase viscosity.

Use naturally occurring polysaccharides listed in Gelling Agents above to aid in emulsifying and gelling. The percentages of these components should not exceed allowable limits in food for each country in which the producted is marketed.

b. Emulsifiers and Cross-linking Agents

To solubilize oil and water components without increasing viscosity. The percentages of these components should not exceed allowable limits in food for each country in which the producted is marketed.

Examples include:

Magnesium salts of fatty acids; Mono- and diglycerides of fatty acids; Acetic acid esters of mono- and diglycerides of fatty acids; Lactic acid esters of mono- and diglycerides of fatty acids; Citric acid esters of mono- and diglycerides of fatty acids; Tartaric acid esters of mono- and diglycerides of fatty acids; Mono- and diacetyltartaric acid esters of mono- and diglycerides of fatty acids; Mixed acetic and tartaric acid esters of mono- and diglycerides of fatty acids Sucrose esters of fatty acids; Sucroglycerides; Polyglycerol esters of fatty acids; Polyglycerol polyricinoleate; Propane-1,2-diol esters of fatty acids; Thermally oxidized soy bean oil interacted with mono and diglycerides of fatty acids; Sodium stearoyl-2-lactylate; Calcium stearoyl-2-lactylate; Stearyl tartrate; Sorbitan monostearate; Sorbitan tristearate; Sorbitan monolaurate; Sorbitan monooleate; Sorbitan monopalmitate; and Invertase.

c. Amino Acids

To increase osmotic pressure, nourish cells, and help bind Ca, Na and K. Ideally, choose the concentrations of each found in the blood of healthy individuals. The percentages of these components should not exceed allowable limits in food for each country in which the producted is marketed.

Examples include:

Alanine; Arginine; Asparagine; Aspartate; Cysteine; Glutamate; Glutamine; Glycine; Histidine; Isoleucine; Leucine; Lysine; Methionine; Phenylalanine; Proline; Serine; Threonine; Tryptophan; Tyrosine; and Valine.

d. Dicarboxylic Acids

To enhance viscosity, nourish cells, and help bind Ca, Na and K.

May choose none to all of the dicarboxylic acids below, but the total of dicarboxylic acids should be less than 1% by weight of the final product to prevent binding of nutrients needed by the cells. Ideally, the concentrations of each found in the blood of healthy individuals should be chosen. The percentages of these components should not exceed allowable limits in food for each country in which the producted is marketed.

Examples include:

Adipic acid; Azelaic acid; Citrate; Fumarate; Glutaric acid; Malate; Oxaloacetate; Sebacic acid; Suberic acid; Succinate; and Tartaric acid.

e. Sugars

To increase osmotic pressure, nourish cells, and for use in glycoform synthesis.

May choose none to all of the sugars below. Ideally, should choose the concentrations of each found in the blood of healthy individuals. The percentages of these components should not exceed allowable limits in food for each country in which the producted is marketed.

Examples include:

L-Arabinose; D-ribose; D-xylose; D-ribulose; 2-deoxy-D-ribose; D-galactose, D-glucose; D-mannose; D-fructose; L-fucose; L-rhamnose; D-mannoheptulose; D-altroheptulose; Glucuronate; Glycerol; Fructose; Mannose; and Fucose.

f. Preservative Sugars

To control microbes and increase osmotic pressure.

The total of these sugars by weight in the formula should be less than 5% or less than 10 g per oral dose. Too much will cause softening of the stools. However, it is recommended that children have at least 5 g a day orally to reduce otitis media (ear infection).

Examples include:

Mannitol; Sorbitol; and Xylitol.

### D. Sources of Phosphate (Any Food Ingredient Containing Phosphate Without Na, K, Ca).

To buffer wound care products and enhance Ca-binding of the dressing.

Use monobasic and dibasic ammonium phosphate to buffer the hydrating agent described in “A” above to a pH of about 7.0 to 7.2 after the amount of hydrating agent needed is determined. The total amount of buffer used should be less than 1% of the total weight of the product. The percentages of these components should not exceed allowable limits in food for each country in which the producted is marketed.

Examples include:

Magnesium phosphate di- and tri-basic; Ammonium phosphate monobasic; Ammonium phosphate dibasic; and Aluminum phosphate.

### E. Food-Grade pH Adjusters (Amino Acids or Dicarboxylic Acids to Lower pH and Provide Amino Acids and Dicarboxilic Acid as Nutrients).

These pH adjusters can be used to adjust the pH of the final product to the desired pH (7-7.4) which is slightly less than the pH of blood. These two ingredients can be combined to make an excellent buffer that maintains the pH of the dressing at a range of 7.0 to 7.4, the preferred range; and 7.25 is optimal. A workable range is pH of 6.0-8.0. The pH should never be lower than 4.0 nor greater than 11.0. Also, the amounts used cannot exceed GMP guidelines.

Magnesium oxide to raise pH and provide magnesium as a nutrient.

Magnesium phosphate di- and tri-basic to provide a phosphate-buffered solution and lower the pH.

NaOH can be used to raise the pH to approximately 14.0.

HCl can be used to lower the pH to approximately 1.0.

### F. Essential Oils (Any Natural Food-Grade Flavor or Fragrance).

Examples include:

Orange oil; Lemon oil; Agar oil; Ajwain oil; Angelica root oil; Anise oil; Balsam oil; Basil oil; Bergamot oil; Black Pepper; Buchu oil; Cannabis flower essential oil; Caraway oil; Cardamom seed oil; Carrot seed oil; Carvacrol; Cedarwood oil; Chamomile oil; Cinnamon oil; Cinnamaldehyde; Cistus; Citronella oil; Clary Sage; Clove oil; Clove leaf oil; Coriander; Costmary; Cranberry seed oil; Cumin oil; Cypress; Davana oil; Dill oil; Eugenol; Fennel seed oil; Fenugreek oil; Fir; Frankincense oil; Galbanum; Geranium oil; Ginger oil; Goldenrod; Grapefruit oil; Henna oil; Helichrysum; Hyssop; Idaho Tansy; Jasmine oil; Juniper berry oil; Laurus nobilis; Lavender oil; Ledum; Lemon oil; Lemongrass; Litsea cubeba oil; Marjoram; Melaleuca; Melissa oil (Lemon balm); Mentha arvensis oil; Menthol; Mountain Savory; Mugwort oil; Mustard oil; Myrrh oil; Myrtle; Nutmeg; Orange oil; Oregano oil; Orris oil; Palo Santo; Parsley oil; Patchouli oil; Perilla essential oil; Perillaldehyde; Pennyroyal oil; Peppermint oil; Petitgrain; Pine oil; Ravensara; Red Cedar; Roman Chamomile; Rose oil; Rosehip oil; Rosemary oil; Rosewood oil; Sage oil; Sandalwood oil; Sassafras oil; Savory oil; Schisandra oil; Spearmint oil; Spikenard; Spruce; Star anise oil; Tangerine; Tarragon oil, distilled from Artemisia dracunculus; Tea Tree oil (Melaleuca oil); Thyme oil; Tsuga; Valerian; Vetiver oil; Western red cedar; Wintergreen; Yarrow oil; and Ylang-ylang.

### G. Preservatives (Any Natural Source Preservatives).

Use any food-grade preservatives as needed to enhance antimicrobial properties so the product can pass the preservative challenge test. Preferred forms are those that do not contain Na, Ca or K ions. Use according to good manufacturing practice guidelines. The percentages of these components should not exceed allowable limits in food for each country in which the producted is marketed.

Examples include:

Methyl paraben; Benzoic acid; Sorbic acid; and Acetic acid.

### H. Vitamins/Cofactors

Zero to all may be used at levels found in healthy people's blood to enhance nutrition for the wound. Do not exceed the concentrations naturally found in blood. The percentages of these components should not exceed allowable limits in food for each country in which the producted is marketed.

Examples include:

Ascorbate; Vitamin D; Vitamins B; Vitamin E; Vitamin K; Vitamin A; and Biotin.

One of skill in the art would recognize that other vitamins and cofactors can be used.

### I. Minerals

Zero to all may be used at levels found in healthy people's blood to enhance nutrition for the wound. Use minerals bound to either amino acids or sugars. Do not exceed the concentrations naturally found in blood. The percentages of these components should not exceed allowable limits in food for each country in which the producted is marketed.

Examples include:

Zinc; Magnesium; Cobalt; Copper; and Selenium.

One of skill in the art would recognize that other minerals can be used.

### General Manufacturing Guidelines for Rationally Designed Wound Care Products

A preferred embodiment of the current invention may include a wound care product comprising an effective amount of a gelling agent and an essential oil from a food, wherein the essential oil has a final concentration that is less than or equal to a concentration of the essential oil found in the food.

1. All the ingredients listed in the patent are allowed for use in the United States. Most of the ingredients are allowed in the rest of the world; however, before making choices, it is useful to refer to the chosen country's good manufacturing practice guidelines for food and wound care products and choose from that country's allowable ingredients.

2. Use ingredients commonly eaten in the country for which the product is intended to be used so that any potential antigens will have a higher likelihood of being turned into tolerogens as a result of previous consumption.

3. It is recommended that oral wound care products containing the ingredients be introduced prior to introducing topical wound care products with the same ingredients. Ideally, a person who has routinely used an oral product to prevent gingivitis would be less likely to have a reaction later when they may use a topical product with similar ingredients. This is a benefit of “letting your food be your medicine”, or at least your dental products.

4. Vitamins and minerals can be added depending on the known deficiencies of a population for which the product is intended.

5. Heating may facilitate gelling of the uniform wound gel product.

6. Most gelling agents can be safely heated to 80 degrees C. This will produce a uniform product faster, and pasteurize the product.

7. Solutions described below may be heated to boiling for at least 20 minutes (if a sterile product is desired). A few vitamins may be deactivated with heat. Consider allowable temperatures before heating vitamins.

8. Fill and package the wound gel product under GMPs required in the country for which the product is intended to be marketed.

9. Always mix the essential oil with the gelling agent before it is added to the hydrating agent. This allows the essential oil to bind to the hydrophobic binding sites on the gelling agent before the hydrophilic binding sites are occupied. For example, if a cotton rag (made of polysaccharide fibers) is first dipped in water, one cannot wipe up oil with that rag; if it's first dipped in oil, one cannot wipe up water with it. For best results, the oils should be mixed with the gelling agent in a sealed container which allows the oil to diffuse throughout the gelling agent and find the unoccupied hydrophobic binding sites. The gelling agent/oil mixtures can be mixed and allowed to stand for months before to use. Gelling agents, particularly those containing polysaccharides, are bound by bacteria. By coating the polysaccharide's hydrophobic binding sites with essential oils, one can concentrate the amount of oil that will come in contact the microorganism. When these sites on a microorganism's surface are occupied by essential oils, the oils kill the bacteria. Essential oils in plants are used to control microorganisms. The polysaccharide gelling agent/oil mixture works like a “gel trap”. The polysaccharide holds the microorganism so the oil can be transferred to its surface, and the microorganism is then killed when the adenosine triphosphate (ATP) is drained from it.

10. Do not use higher concentrations of essential oils than those found in the foods eaten by the population for which the product is intended. Essential oils are found in edible plants at concentrations of from 1-13%.

11. Vitamins, minerals, monosaccharides, amino acids and dicarboxylic acids can be roller compacted with gelling agents and re-ground for use in Mixing Method 2 described below.

12. One of skill in the art would recognize that other ingredients than the ones listed above may be used to create products covered by this patent. The recommended pH and osmotic pressure are intended as general guidelines; however, rational exceptions can be made for special use products.

13. Beta-bonded gelling agents are preferred for all oral products to prevent amylase degradation of the gel into monosaccharides, which can cause caries.

14. All dental products should contain essential oils, such as carvacrol or eugenol, to enable the gel to pass the preservative challenge test and control the microbes and viruses responsible for dental caries, gingivitis and periodontitis.

### Mixing Methods

### Mixing Method 1

### Solution 1

To prepare the hydrogel, begin by weighing one or more cross-linking and cation-binding agents, preservatives, vitamins/cofactors, and minerals. Add these ingredients to a hydrating agent. Mix until all components are in solution. Buffer with one or more sources of phosphate to a pH of 7.2. It is preferred that the sources of phosphate not exceed 1% of the final product. If this is not possible, obtain the desired pH by using the maximum desirable amount of sources of phosphate (1%), use food-grade pH adjusters to obtain the desired pH of 7.2. (In the pilot manufacturing steps, make sure that Solution 1 has enough food grade preservatives and alcohol sugars to pass the preservative challenge test before Mixture 1 is added.)

### Mixture 1

Blend a gelling agent and one or more natural flavors together. If needed, blend natural flavors with ethanol and then blend with gelling agent to achieve a uniform mixture. (Usually, addition of ethanol is not needed.) Allow the weight of ethanol that was added to evaporate; this produces a gelling agent uniformly coated with the flavors.

Add mixture 1 to solution 1 and mix until a uniform product is formed.

Before filling and packaging, measure the pH of the uniform wound gel product. If necessary, use one or more food-grade pH adjusters to readjust the room temperature product to a pH of approximately 6 to 8, preferably a pH of 7.2. This step can be avoided by adjusting the pH of Solution 1 up or down so that the final product will have a pH of 7.2. For example, if the final product has a pH of 7, solution 1 can be adjusted to a pH of 7.4 initially and it will usually result in a product with a pH of 7.2. By repeating this adjustment in the pilot manufacturing steps, one can avoid the need to adjust the final product. Despite the fact that specific pH is given here, one of skill in the art would recognize that pH can be adjusted within physiologically compatible ranges.

### Mixing Method 2

### Solution 2

Buffer the desired amount of a hydrating agent with one or more sources of phosphate to a pH of 7.2.

### Mixture 2

Mix the desired gelling agent with the desired amounts of one or more essential oils, vitamins/cofactors, minerals, amino acids, dicarboxylic acids, and sugars as described in mixture 1. Roller compact and finely grind this mixture.

Then add mixture 2 to solution 2 and mix until a uniform product is formed.

Before filling and packaging, measure the pH of the uniform wound gel product. Use food-grade pH adjusters to readjust the room temperature product to a pH of 7.2. This step can be avoided by adjusting the pH of Solution 1 up or down so that the final product will have a pH of 7.2. For example, if the final product has a pH of 7, one could have initially adjusted solution 1 to a pH of 7.4, and it would usually result in a product with a pH of 7.2. By repeating this adjustment in the pilot manufacturing steps, one can avoid the need to adjust the final product. One of skill in the art would recognize that there are physiologically acceptable pHs of other than 7.2.

### Mixing Method 3

One of skill in the art would recognize that other methods can be used.

### Using the Hydrogel Wound Dressing

This invention is applied to the wound in the same way current hydrogel dressings are used by health care practitioners.

Differentiating it from Other Analgesic Wound Dressings

This invention describes an analgesic hydrogel wound dressing composed entirely of food ingredients and that does not contain a drug of any kind. In contrast to topical analgesic drugs, this hydrogel breaks down into nutrients that are useful to the body, rather than into drugs that can be harmful. It can be standardized and manufactured under GMPs for commercial distribution. Topical drugs work by blocking ion channels, a generalized effect. In contrast, this present invention works by mechanically trapping ions of Ca, Na and K in the hydrogel at the wound site so the sensation of pain cannot be transmitted. It breaks down into nutrients that are useful to the body, as described in the section titled “summary of the general idea” above.

### Preferred Compositions

The following preferred compositions can be used to produce an effective product by both Mixing Methods 1, 2 and 3.

The products must be manufactured under Good Manufacturing Practices (GMPs) in the United States and must be manufactured under International Standards Organization (ISO) or equivalent standards in the rest of the world. These products by law must be manufactured by persons skilled in one or both standards, because each country has their own unique guidelines for use of these ingredients. The law requires that one uses current standards which may be changed regularly, therefore, whoever manufactures products taught by this patent must follow GMPs or ISO standards for the country in which the product will be marketed.

For example the following formula is used to produce a dental wound gel (“SOCK IT®”), intended to by used after brushing the teeth, left in the mouth, and swallowed. Therefore, one must use GMP guidelines or equivalent standards that are specific for food ingredients because one does not want to exceed the allowable limits for food. This formula will make a 100.0 gram or three and one-third (3⅓) oz. tube. The GMP guidelines (in the United States) are cited for this product. Any ingredients not allowed in specific countries are not to be used. The % used herein denotes weight %.

Regulatory Status of Ingredients as Foods

| | | Percent | Maximum |
|---|---|---|---|
| | | Allowed | % in |
| Ingredient | Regulatory Status | in Food | SockIt! |
| Brown algae {Undaria pinnatifida} | GRAS/21 CFR 184.1120 | GMP | 5.00% |
| (flavoring and thickening agent) | | | |
| Xylitol (flavoring) | Food Additive/21 CFR 172.395 | GMP | 0.92% |
| Sorbitol (flavoring) | GRAS/21 CFR 184.1835 | ≧99% | 0.92% |
| Trans-cinnamaldehyde (flavoring) | GRAS/21 CFR 182.60 | GMP | 0.50% |
| Carvacrol (flavoring) | Food Additive/21 CFR 172.515 | GMP | 0.50% |
| Eugenol (flavoring) | GRAS/21 CFR 184.1257 | GMP | 0.50% |
| Menthol (flavoring) | Food Additive/21 CFR 172.515 | GMP | 0.50% |
| | and 182.20 | | |
| Thymol (flavoring) | Food Additive/21 CFR 172.515 | GMP | 0.50% |
| | and 175.105 | | |
| Ammonium phosphate | GRAS/21 CFR 184.1141.a | GMP | 0.0083% |
| monobasic | | | |
| (pH adjuster) | | | |
| Ammonium phosphate | GRAS/21 CFR 184.1141.b | GMP | 0.016% |
| dibasic (pH adjuster) | | | |
| Tartaric acid (pH adjuster and | GRAS/21 CFR 184.1099 | GMP | 0.016% |
| firming agent) | | | |
| Water (solvent) | GRAS | No limitation | 90.4497% |

### Preferred Composition 1

Mix cinnamaldehyde (0.1%), carvacrol (0.05%), eugenol (0.15%), menthol (0.1%), and thymol (0.1%). Pour this mix onto the undaria (2.5%) and blend to obtain Mixture 1. Dissolve tartaric acid (0.016%), ammonium phosphate monobasic (0.008%), ammonium phosphate dibasic (0.016%), xylitol (1%), and sorbitol (1%) in water (94.992%). The pH of this solution (Solution 1) should be 7.4. Add Mixture 1 to Solution 1 and mix until a uniform product is formed.

### Preferred Composition 2

Mix cinnamaldehyde (0.1%), carvacrol (0.05%), eugenol (0.15%), menthol (0.1%), and thymol (0.1%). Pour this mix onto the undaria (0.5%), konjac gum (1.25%), xanthan gum (0.05%) and tara gum (1.25%) that were previously mixed, and blend to obtain Mixture 1. Dissolve tartaric acid (0.016%), ammonium phosphate monobasic (0.008%), ammonium phosphate dibasic (0.016%), xylitol (0.92%), and sorbitol (0.92%) in water (95.02%). The pH of this solution (Solution 1) should be 7.4. Add Mixture 1 to Solution 1 and mix until a uniform product is formed.

### Preferred Composition 3

Mix cinnamaldehyde (0.1%), menthol (0.1%), and thymol (0.1%). Pour this mix onto the undaria (3%) and xanthan gum (0.05%) that were previously mixed, and blend to obtain Mixture 1. Dissolve ammonium phosphate monobasic (0.008%), ammonium phosphate dibasic (0.016%), xylitol (0.92%), and sorbitol (0.92%) in water (94.786%). The pH of this solution (Solution 1) should be 7.4. Add Mixture 1 to Solution 1 and mix until a uniform product is formed.

### Preferred Composition 4

Mix cinnamaldehyde (0.1%), carvacrol (0.05%), eugenol (0.2%). Pour this mix onto the konjac gum (0.6%) and tara gum (0.6%) that were previously mixed, and blend to obtain Mixture 1. Dissolve ammonium phosphate monobasic (0.008%), ammonium phosphate dibasic (0.039%) and xylitol (2%) in water (96.403%). The pH of this solution (Solution 1) should be 7.4. Add Mixture 1 to Solution 1 and mix until a uniform product is formed.

### Preferred Composition 5

Mix cinnamaldehyde (0.15%) and eugenol (0.15%). Pour this mix onto the konjac gum (0.8%) and tara gum (0.8%) that were previously mixed, and blend to obtain Mixture 1. Dissolve ammonium phosphate monobasic (0.008%), ammonium phosphate dibasic (0.016%), mannitol (0.92%), and sorbitol (0.92%) in water (96.236%). The pH of this solution (Solution 1) should be 7.4. Add Mixture 1 to Solution 1 and mix until a uniform product is formed.

### Preferred Composition 6

Mix cinnamaldehyde (0.15%), carvacrol (0.05%), and thymol (0.1%). Pour this mix onto the locust bean gum (1%), xanthan gum (1%) and guar gum (0.5%) that were previously mixed, and blend to obtain Mixture 1. Dissolve ammonium phosphate monobasic (0.008%), ammonium phosphate dibasic (0.016%), xylitol (0.92%), and mannitol (0.92%) in water (95.336%). The pH of this solution (Solution 1) should be 7.4. Add Mixture 1 to Solution 1 and mix until a uniform product is formed.

### Preferred Composition 7

Mix carvacrol (0.15%) and eugenol (0.2%). Pour this mix onto the undaria (1.25%) and konjac gum (1.25%) that were previously mixed, and blend to obtain Mixture 1. Dissolve ammonium phosphate monobasic (0.008%), ammonium phosphate dibasic (0.016%), xylitol (0.92%), and sorbitol (0.92%) in water (95.286%). The pH of this solution (Solution 1) should be 7.4. Add Mixture 1 to Solution 1 and mix until a uniform product is formed.

### Preferred Composition 8

Mix cinnamaldehyde (0.05%), wintergreen oil (0.1%), eugenol (0.2%), ethanol (0.3%). Pour this mix onto the xanthan gum (0.5%) and tara gum (0.5%) that were previously mixed, and blend to obtain Mixture 1. Dissolve ammonium phosphate monobasic (0.008%), ammonium phosphate dibasic (0.04%), mannitol (0.92%), and sorbitol (0.92%) in water (96.462%). The pH of this solution (Solution 1) should be 7.4. Add Mixture 1 to Solution 1 and mix until a uniform product is formed.

### Preferred Composition 9

Pour eugenol (0.1%) onto the tara gum (1.0%) to obtain Mixture 1. Dissolve ammonium phosphate monobasic (0.008%), ammonium phosphate dibasic (0.04), xylitol (2.0%) and water (96.852). The ph of this solution (Solution 1) should be 7.4. Add Mixture 1 to Solution 1 and mix until a uniform product is formed.

### Preferred Composition 10

Mix sandalwood oil (0.05%), wintergreen oil (0.1%), eugenol (0.2%), ethanol (0.3%). Pour this mix onto the xanthan gum (0.5%) and tara gum (0.5%) that were previously mixed, and blend to obtain Mixture 1. Dissolve ammonium phosphate monobasic (0.008%), ammonium phosphate dibasic (0.04%), mannitol (0.92%), and sorbitol (0.92%) in water (96.462%). The pH of this solution (Solution 1) should be 7.4. Add Mixture 1 to Solution 1 and mix until a uniform product is formed.

### Preferred Composition 11

Use essential oils commonly found in food of the population for which the product is intended and in a concentration equal to or lower than that found naturally in food. Pour this mix onto the xanthan gum (0.5%) and tara gum (0.5%) that were previously mixed, and blend to obtain Mixture 1. Dissolve ammonium phosphate monobasic (0.008%), ammonium phosphate dibasic (0.04%), mannitol (0.92%), and sorbitol (0.92%) water (QS to 100%). The pH of this solution (Solution 1) should be 7.4. Add Mixture 1 to Solution 1 and mix until a uniform product is formed.

### Preferred Composition 12

Use essential oils commonly found in food of the population for which the product is intended and in a concentration equal to or lower than that found naturally in food. Pour this mix onto a mixture of sufficient gelling agents as to allow extrusion into sheets that could be applied as strips of varying sizes to the wound. Dissolve ammonium phosphate monobasic (0.008%), ammonium phosphate dibasic (0.04%), mannitol (0.92%), and sorbitol (0.92%) water (QS to 100%). The pH of this solution (Solution 1) should be 7.4. Add Mixture 1 to Solution 1 and mix until a uniform extrudable product is formed.

### Efficacy of Products in the Dental Applications:

The use of compositions by dental patients with various oral injuries, after dental procedures, stated that the hydrogel wound dressing compositions in SOCK IT® were able to manage pain for an extended period of time. Additionally, numerous patients were able to reduce their intake of additional pain medications. Furthermore, dental professionals stated that less cases of infection were visible in post-opt visits due to the prevention of moisture loss and creation of a barrier between the wound and exogenous debris. Several dentists have reported that the incidence of dry socket cases diminished significantly, when the composition was used as part of the treatment regimen. Here are some testimonies:

“I found that I needed no pain medication. The gel combated discomfort . . . with surprising effect for up to 6 hours.”—45-year-old female. 25 teeth extracted-immediate dentures.

“Worked well. Did not need the pain pills prescribed by the doctor. Did not even need over-the-counter medication.”—77-year-old male; lower bony impacted 3^{rd} molar extraction.

“It worked just great. The pain was gone in seconds and it lasted for hours.”—51-year-old female; deep scaling procedure with no relief from prescription medication

“It caused a mild stinging for about 10 second, then all the pain would be gone for more than 4 hoours.”—52-year-old female; alhthous ulcer

“The gel has been a life save allowed me to go back to my sales job the day after surgery.”—37-year-old female; 2 teeth extracted

“The dentist used the gel after they finished cleaning my teeth. After the numbness wore off I had no pain. I used it every 4 hours that day and never felt any pain.”—60-year-old male; deep tooth scaling

Parents have reported it is the best teething gel which they have used on their children, not only does it control pain, and the taste is not offensive to most children.

Adults have reported that the compositions are able to control pain associated with oral wounds without causing a numbing sensation at the site of application.

Doctors' offices have reported that application of the composition on fireant bites stopped the pain and promoted healing. Furthermore, this effect is also reported when it is applied to other bites ands stings from wasps, bees, and spiders.

Patents with past drug dependency loved the product because they do not have to take pain drugs, and therefore do not have to worry about any possible recurrent dependency.

Physical and Chemical Properties of Compositions which are Ideal for Wound Care Products:

Any compositions created according to this patent are able to create hydrogel solutions that can be applied orally or topically, which is not the case for most commercial wound dressings. This is partly due to the lack of common artificial preservatives, which proved toxic upon ingestion and cytotoxic to various beneficial cells needed for wound healing and repair.

Hydrogel wound dressings designed in accordance with this patent create a stable gel which able to create a barrier between the wound and the external environment. The composition also serves to maintain optimal moisture at the wound site, which is conclusive to wound healing and repair. Additionally, various components in the compositions serve to manage pain by sequestering and holding the ions implicated in the transduction of pain signals. Furthermore, other ingredients in the composition serve to prevent and control infection by eliminating microorganismal growth and propagation.

Any wound care products designed in accordance with this patent would be able to maintain optimal moisture at the wound site, control pain, prevent microbial infection, and would not induce the death of health cells and tissues. Clinical trials underway are demonstrating that these compositions are helpful to all oral wounds and that it appears to accelerate healing and control pain.

### REFERENCES CITED

The following references, to the extent that they provide exemplary procedural or other details supplementary to those set forth herein, are specifically incorporated herein by reference.
(1) Ovington L G. Advances in wound dressings. Clin Dermatol. 2007; 25:33-38.
(2) Motta G. WOUNDSOURCE: The Kestrel wound product sourcebook. Kestrel Health Information Inc, 2005.
(3) Jacob S E, James W D. From road rash to top allergen in a flash: bacitracin. Dermatol Surg. 2004; 30:521-524.
(4) Gore M A, Akolekar D. Evaluation of banana leaf dressing for partial thickness burn wounds. Burns. 2003; 29:487-492.
(5) Gore M A, Akolekar D. Banana leaf dressing for skin graft donor areas. Burns. 2003; 29:483-486.
(6) Ingle R, Levin J, Polinder K. Wound healing with honey—a randomised controlled trial. S Afr Med J. 2006; 96:831-835.
(7) Subrahmanyam M. Topical application of honey in treatment of burns. Br J Surg. 1991; 78:497-498.
(8) Okeniyi J A, Olubanjo O O, Ogunlesi T A, Oyelami O A. Comparison of healing of incised abscess wounds with honey and EUSOL dressing. J Altern Complement Med. 2005; 11:511-513.
(9) FDA. Warning Letter to Trianle Compounding Pharmacy. Dec. 4, 2006.
   Ref Type: Personal Communication
(10) FDA. Warning Letter to University Pharmacy. Dec. 4, 2006.
   Ref Type: Personal Communication
(11) Kandel E, Jessell T, Schwartz J. Principles of Neural Science. 4, Revised, illustrated ed. McGraw-Hill Professional Publishing; 2000.
(12) Willis W, Coggeshall R. Sensory Mechanisms of the Spinal Cord. 3, illustrated ed. Springer; 2004.
(13) Oh U, Benos D, Simon S. The Nociceptive Membrane. illustrated ed. Elsevier Science & Technology Books; 2006.
(14) Blankenship J. Neurophysiology. 2003.
(15) Catterall W A. Structure and function of voltage-sensitive ion channels. Science. 1988; 242:50-61.
(16) Nolte J. The Human Brain: An Introduction to Its Functional Anatomy. 5, Revised, illustrated ed. Mosby, Incorporated; 2002.
(17) Harman J, Limbird L, Goodman L, Gilman A. Goodman and Gilman's the Pharmacological Basis of Therapeutics. McGraw-Hill Professional Publishing; 2001.
(18) Teughels W, Sliepen I, Quirynen M et al. Human cytomegalovirus enhances A. actinomycetemcomitans adherence to cells. J Dent Res. 2007; 86:175-180.
(19) Slots J. Herpesviral-bacterial synergy in the pathogenesis of human periodontitis. Curr Opin Infect Dis. 2007; 20:278-283.
(20) Playfair J H L, Chain R. Immunology at a Glance. 7th ed. Blackwell Science; 2001.
(21) United States Pharmacopeia—National Formulary.
(22) Dorland's Medical Dictionary for Health Consumers.© 2007 by Saunders, an imprint of Elsevier, Inc.

What is claimed:

1. A wound care product comprising an effective amount of each of the following:
a gelling agent; and
an essential oil from a food,
wherein the essential oil has a final concentration that is less than or equal to a concentration of the essential oil found in the food.

2. The wound care product of claim 1, wherein the essential oil is sandalwood, lavender, orange, cedar, jasmine, peppermint, rosemary, sage oi or a mixture thereof.

3. The wound care product of claim 1, further comprising a hydrating agent.

4. The wound care product of claim 1, further comprising a cross-linking agent.

5. The wound care product of claim 1, further comprising a cation-binding agent.

6. The wound care product of claim 1, wherein the wound care product further comprises fragrant essential oils or other fragrant ingredients.

7. The wound care product of claim 1, wherein the wound care product further comprises a beta-bonded food-grade polysaccharide.

8. The wound care product of claim 1 wherein the gelling agent and the essential oil from the food are of food ingredients or food additives.

9. The wound care product of claim 1, further comprising a preservative.

10. The wound care product of claim 1, further comprising a vitamin or cofactor.

11. The wound care product of claim 1, further comprising a mineral.

12. The wound care product of claim 1, further comprising a source of phosphate.

13. The wound care product of claim 1, further comprising beta-linked mannans.

14. The wound care product of claim 1, further comprising a natural flavor or food-grade flavoring agent.

15. The wound care product of claim 1, further comprising a buffer.

16. The wound care product of claim 1, wherein the wound care product has a pH value adjusted to a pH of about 7.2.

17. The wound care product of claim 1, wherein the wound care product provides a protective barrier from further irritation and insult to the wound.

18. A method for treating a wound in a subject, comprising:
applying the wound care product of claim 1 to the subject.

19. The method of claim 18, wherein the wound is selected from the group consisting of physical burns, chemical burns, chapped lips, partial thickness skin grafts, full thickness skin grafts, skin flaps, biopsy sites, excision biopsy sites, punch biopsy sites, shave biopsy sites, fine needle aspiration sites, suture sites, suture removal sites, staple sites, staple removal sites, wounds closed with adhesive compounds, wounds closed with adhesive strips, wounds closed by secondary intention, tattoos, areas treated with lasers, areas treated with Intense Pulsed Light, areas treated with chemical peals, areas treated with dermabrasion, areas treated with micro-dermabrasion, areas of hair transplants, dermatitis, intravenous catheter sites, cutaneous penetration site of drains including Jackson-Pratt and Penrose, cuntaneous penetration site of chest tubes, injection sites, immunization sites, insulin injection sites, intramuscular injection sites, sites of local anesthetic administration, sites of injection of anticoagulants, sites of injection of cosmetic paralytics including BOTOX® (botulinum toxin), sites of injection of cosmetic filling and contouring agents including Restylane® (hyaluronic acid), Sculptra™ (poly-L-lactic acid) and Radiesse™ (calcium hydroxylapatite), sclerotherapy injections, mesotherapy injections, cutaneous penetration site of all transcutaneous internal fixation devices, cutaneous penetration site of all transcutaneous external fixation devices, wounds obtained during piercing, diabetic ulcers, foot ulcers, pressure ulcers (stage 1-4), skin conditions associated with peristomal care, radiation dermatitis, sunburn, cuts and abrasions.

20. The use of the wound care product of claim 1 for use in aphthous ulcers, extraction site pain, pain of dry sockets, oral mucositis and stomatitis, pain following tooth scaling and prophylaxis, pain of gingivitis, all types of surgical wounds, soft tissue pain from orthodontics, irritation and traumatic ulcers such as those caused by various appliances such as braces, brackets, full and partial dentures and palatal expanders, management of teething pain in infants, pain associated with sensitization of teeth and gums, e.g. from use of teeth whiteners and other dental cosmetics, or hemorrhoids.

21. The use of the wound care product of claim 1 for treatment of oral wounds, mouth sores, injuries or ulcers of the oral mucosa.

22. A wound care product comprising the following ingredients:
cinnamaldehyde, eugenol, menthol, thymol blended in undaria; tartaric acid, ammonium phosphate monobasic, ammonium phosphate dibasic, xylitol, and sorbitol in water;
wherein the ingredients have been mixed until a uniform product is formed.

23. A wound care product comprising the following ingredients:
cinnamaldehyde, carvacrol, and eugenol, blended in konjac gum and tara gum;
ammonium phosphate monobasic, ammonium phosphate dibasic, and xylitol in water;
wherein the ingredients have been mixed until a uniform product is formed.

24. A wound care product comprising:
cinnamaldehyde and eugenol, blended in konjac gum and tara gum; and
ammonium phosphate monobasic, ammonium phosphate dibasic, mannitol, and sorbitol in water;
wherein the ingredients have been mixed until a uniform product is formed.

25. A wound care product comprising:
sandalwood oil, wintergreen oil, eugenol, ethanol blended in xanthan gum and tara gum;
ammonium phosphate monobasic, ammonium phosphate dibasic, mannitol, and sorbitol in water;
wherein the ingredients have been mixed until a uniform product is formed.

26. A wound care product comprising:
essential oils mixed with a gelling agent;
ammonium phosphate monobasic, ammonium phosphate dibasic, mannitol, and sorbitol in water;
wherein the ingredients have been mixed until a uniform product is formed.
